(19) United States
(12) Patent Application Publication
(10) Pub. No.: US 2018/0117331 A1
(43) Pub. Date: May 3, 2018

(54) Minimally Invasive Subgaleal Extra-Cranial Electroencephalography EEG Monitoring Device

(71) Applicant: New York University, New York, NY (US)

(72) Inventors: Ruben I. KUZNIECKY, Englewood, NJ (US); Werner K. Doyle, New York, NY (US); Steve Pacia, New York, NY (US); Daniel Friedman, New York, NY (US)

(21) Appl. No.: 15/342,873

(22) Filed: Nov. 3, 2016

(51) Int. Cl. A61N 1/36; A61B 5/0476; A61B 5/00

(52) U.S. Cl. CPC A61N 1/36064; A61B 5/0476; A61N 1/36135; A61B 5/6868; A61B 5/4094

(57) ABSTRACT

A system includes an implantable body configured for implantation in a subgaleal extracranial position, the implantable body including a first electrode array including a first elongated body comprising first and second electrode contacts separated from one another by a distance selected to facilitate the detection of brain electrical activity and a unit coupled to the first electrode array. The unit includes a processor analyzing the detected brain electrical activity to determine whether an epileptic event has occurred and generating epileptic event data based on this determination and a transceiver controlled by the processor to wirelessly transmit epileptic event data to and from a remote computing device.

### BACKGROUND

For epilepsy patients, an objective seizure detection method that is safe, accurate and does not interfere with patient activities is critical to advance patient care. Seizure frequency is the most important index for determining and monitoring seizure control. Unfortunately, many patients experiencing seizures lose consciousness or are amnesic while most subtle or non-convulsive seizures may be unobserved or unrecognized. As the majority of current determinations of seizure frequency are based on patient and/or caregiver reports, these determinations represent, at best, a crude estimate of the true frequency. Studies have shown that seizure counts reported by the patients or caregivers can have an error rate as high as 60% which has important implications for therapy. Unrecognized seizures have a major effect on epilepsy clinical trials as the error rate and placebo effect are driven largely by the subjective nature of seizure count. Furthermore, unrecognized seizures increase the risk of cognitive decline, injury and death.

The only objective method to account for seizures is in-patient video Electroencephalography (EEG) or ambulatory EEG recordings. Video EEG studies are inpatient based and thus very expensive and only used for acute situations or for pre-surgical investigations. Current methods for ambulatory scalp EEG recordings are not practical for long-term use and can typically be used for a maximum of 3-4 days. These methods are also limited due to scalp EEG artifacts, battery life or the inability of patients to tolerate scalp electrodes from more than a few days. Implantation of intracranial EEG electrodes for long-term monitoring has been developed for situations where epilepsy surgery is appropriate. However, not only are these highly invasive procedures expensive (generally more than $50,000), they also have a high morbidity rate. Such procedures are indicated only for patients who are candidates for resective epilepsy surgery yet there are vastly more patients who would benefit from such implantable EEG surveillance. Seizure detection systems based on body motion or motor activity, accelerometers or video detectors can only detect major convulsive events and are not implantable, limiting their value as most seizures are non-convulsive in nature and therefore not detectable with changes in motion. There is a long-felt need for a device that can accurately record EEG activity relatively free of artifact for extended periods of time (e.g., months to years) and can detect, recognize and store seizure events in patients without causing discomfort to the patient and without compromising their health and safety.

### SUMMARY OF THE INVENTION

The present invention is directed to a unitary implantable device, comprising an elongated implantable body configured for implantation at or near a cranial vertex in a subgaleal extracranial space of a patient. The device includes a first and a second electrode contacts separated from one another by a distance selected to form a single channel for detection of brain electrical activity. The device also includes a processor analyzing the detected brain electrical activity to determine whether a change in brain state has occurred and generating brain state data based on this determination. The device further includes a transceiver controlled by the processor to wirelessly transmit epileptic event data to and from a remote computer.

In one aspect a method for capturing brain wave data is provided. The method comprises inserting, using a minimally invasive surgical technique, an implantable body into a subgaleal extracranial position at or near a cranial vertex of a patient, the implantable body positioned along a cranial surface at least 1 cm away from the temporalis muscles of the patient, and so that first and second electrode contacts of a first electrode array of the implantable body face the cranium. The method includes detecting brain electrical activity via a single channel formed by the first and second electrode contacts. The method also includes monitoring and analyzing, via a processor, the brain electrical activity to detect epileptic events. The method further includes transmitting epileptic event data corresponding to a detected epileptic event to one of a remote computer, local computer, local base station, cellular phone, portable tablet, personal computing device and cloud storage.

These and other aspects of the invention will become apparent to those skilled in the art after a reading of the following detailed description of the invention, including the figures and appended claims.

### BRIEF DESCRIPTION OF THE DRAWINGS

FIG. 1 shows a schematic drawing of an exemplary system according to the invention;

FIG. 2 shows a perspective view of an implantable device according to a first embodiment of the invention;

FIG. 3 shows a first view of the device of FIG. 2 in an implanted configuration;

FIG. 4 shows a second view of the device of FIG. 2 in the implanted configuration;

FIG. 5 shows a perspective view of an implantable device according to an alternate embodiment of the invention;

FIG. 6 shows a perspective view of an implantable device according to another embodiment of the invention;

FIG. 7 shows a perspective view of an implantable device according to a further embodiment of the invention;

FIG. 8 shows a side view of the device of FIG. 6;

FIG. 9 shows a top view of the device of FIG. 6;

FIG. 10 shows a first view of the device of FIG. 6 in an implanted configuration;

FIG. 11 shows an exemplary view of an exemplary device in an implanted configuration;

FIG. 12 shows EEG data recorded over time by electrodes implanted directly under the dura mater and by an exemplary device implanted into a subgaleal region of a patient;

FIG. 13 shows the EEG data recorded over time in an awake patient who is being evaluated for epilepsy surgery by intracranial electrodes implanted directly under the dura mater as well as EEG data recorded by an exemplary subgaleal device of the present invention;

FIG. 14 shows EEG data recorded over time in an asleep patient who is being evaluated for epilepsy surgery by intracranial electrodes implanted directly under the dura mater as well as EEG data recorded by an exemplary unitary subgaleal device of the present invention;

FIG. 15 shows the EEG data recorded over time in a patient who is being evaluated for epilepsy surgery by intracranial electrodes implanted directly under the dura mater as well as EEG data recorded by an exemplary subgaleal device of the present invention;

FIG. 16 shows EEG data recorded over time in a patient who is being evaluated for epilepsy surgery by intracranial electrodes implanted directly under the dura mater as well as EEG data recorded by an exemplary subgaleal device of the present invention;

FIG. 17 shows an exemplary method for detection of seizure using subgaleal EEG data;

FIG. 18 shows an exemplary embodiment of a decision tree incorporated by the method shown in FIG. 17, which may include a database for containing data and features recorded from numerous patients, including healthy patients as well as patients who suffer from epileptic seizures;

FIG. 19 shows an exemplary embodiment of a single integrated device having six different contacts;

FIG. 20 shows EEG data recorded over time in a patient during inter-ictal periods during sleep by intracranial electrodes implanted directly under the dura mater as well as EEG data recorded by an exemplary subgaleal device of the present invention;

FIG. 21 shows a power density plot of the data of FIG. 20;

FIG. 22 shows a band power density plot of the data of FIG. 20;

FIG. 23 shows EEG data recorded over time in a patient during a period including a complex partial seizure by intracranial electrodes implanted directly under the dura mater as well as EEG data recorded by an exemplary subgaleal device of the present invention;

FIG. 24 shows a power density plot of the data of FIG. 23; and

FIG. 25 shows a band power density plot of the data of FIG. 23.

### DETAILED DESCRIPTION

The present invention is directed to a system and method for recording EEG activity and detecting epileptic events over an extended continuous period of time to aid in the diagnosis and treatment of epilepsy and other neurological conditions. An exemplary device includes one or more electrode arrays configured for subgaleal implantation (i.e., between the galea aponeurotica and the pericranium). In certain embodiments, the one or more electrode arrays may be configured to record EEG activity and/or detect brain states or epileptic events using contacts that detect electromagnetic activity from a position external to the cranium, in particular, the electromagnetic activity (e.g., EEG) may be detected from a subgaleal region of a patient. Thus, while current devices require drilling through the cranium to implant electrodes directly onto the dura mater, into the subdural space or in the parenchyma, the exemplary device according to the invention permits implantation extra-cranially, reducing trauma, bleeding, eliminating the need to expose the brain and significantly limiting adverse side effects and consequences of surgery, as those skilled in the art will understand. Furthermore, for most patients, implantation would occur in the ambulatory surgery setting with minimal recovery time and no inpatient hospital stay and much less cost.

Furthermore, the exemplary device for subgaleal implantation may also provide improved detection of seizure as compared to conventional intracranial electrodes. Although intracranial EEG electrodes are capable of recording directly from brain tissue and therefore, obtaining information directly from the brain and is more accurate for the purpose of localization of seizure activity and temporal sampling, such intracranial EEG electrodes is limited in its scope to small or relatively restricted areas of the brain. In other words, conventional intracranial EEG electrodes cannot sample the entire brain and therefore, may not be able to detect seizure activity unless the electrodes are place in close proximity to the portion of the brain experiencing ictal activity. In contrast, subgaleal EEG electrodes are placed outside the skull. Although the electrophysiological potentials detected by subgaleal EEG may be more attenuated as compared to intracranial EEG electrodes, the subgaleal EEG recordings provide improved spatial resolution for detecting seizure activity as compared to intracranial electrodes. Specifically, if an intracranial EEG electrode is not placed in close proximity to the portion of the brain where the ictal activity occurs, it may not be able to detect the seizure, whereas subgaleal EEG may have a broader spatial range and be capable of detecting the spread of a seizure throughout the brain with much more sensitivity as compared to intracranial EEG. This is further demonstrated in Example III below.

The exemplary electrode strip according to the invention comprises a plurality of contacts separated from one another by a predetermined length selected to provide a predetermine sensitivity reading for detecting ictal activity, as those skilled in the art will understand and as described in greater detail below. The plurality of contacts are formed with one of the same and variable contact surface areas and are arranged in particular patterns, as described in greater detail later on. The electrode strip is a flat, flexible biocompatible array connected to an exemplary battery and electronics compartment configured for long-term (i.e., 12 or more months) implantation. The device further comprises programmable analog and digital electronics configured to monitor long-term brain activity without excessively draining the battery and without excessive power consumption, as will be described in greater detail below. The exemplary device is configured to primarily record electrical brain activity and determine, based on the detected brain activity, whether an epileptic event has occurred. In addition, the device can determine awake versus sleep stages, abnormal EEG patterns that may occur in many brain disorders. Other biometric measurements such as but not limited to blood oxygenation, arterial pulse, galvanic resistance, motion/acceleration, and electro-myographic measurements, anticipated to augment the primary electrical brain activity measurement may also be incorporated into the exemplary device. Epileptic event data corresponding to the detected epileptic events is stored in an electronic memory provided in the implanted device. The device may further comprise a wireless transmitter and receiver (i.e., a transceiver) to transmit the EEG data and/or ictal events data to an external computing device and/or cloud-based data storage and review services. Transmission of data may occur according to one or more patterns (e.g., routine transmission at predetermined time intervals, location-based (e.g., when in proximity to a base station or appropriate personal smart device), upon being prompted to transmit by a secondary device, etc.) If a time-based transmission is used, the rate of transmission may be programmed into the onboard memory. In one example, the data may be programmed for transmission every 1-7 days as determined based on seizure rates estimated by a clinician. Data from the device may be transmitted to the external computing device periodically to reduce the required memory. The exemplary device according to the invention is adapted for implantation at a cranial vertex or a vicinity thereof, as will be described in greater detail below. It is noted, however, that the other implantation locations on the head are also envisioned within the scope of the invention.

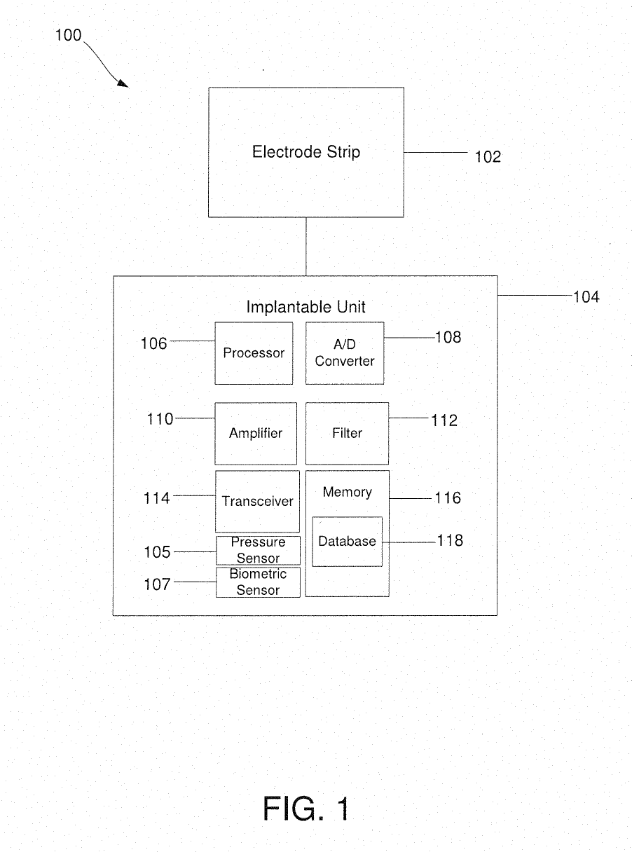

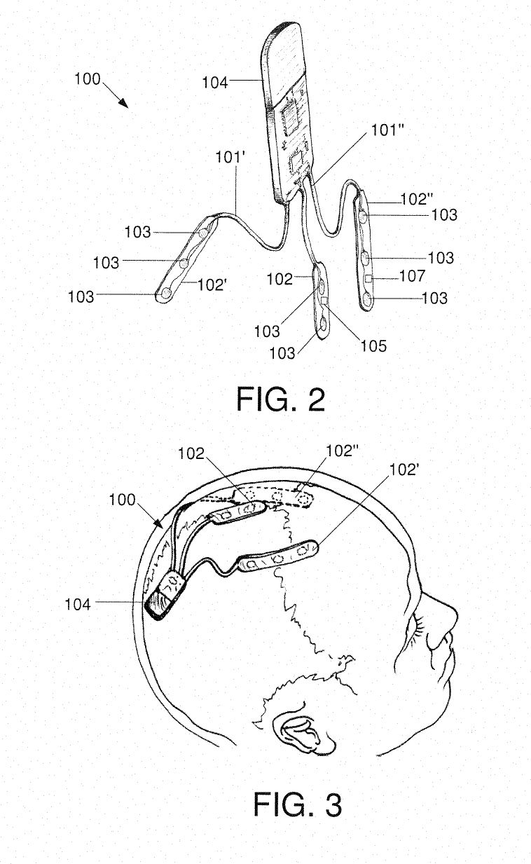

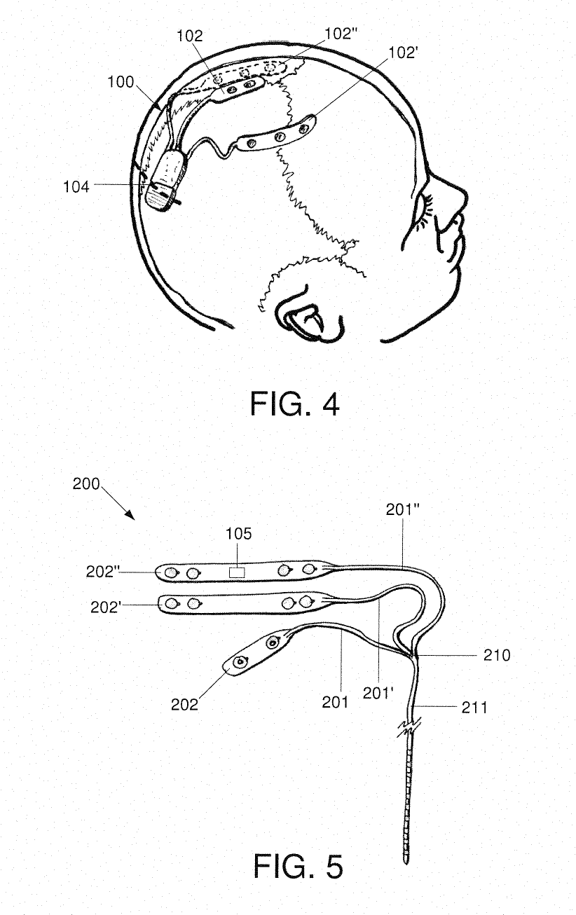

As shown in FIGS. 1-4, an exemplary device 100 according to the invention is dimensioned to permit subgaleal insertion and comprises an implantable unit 104 coupled to one or more low profile electrode arrays 102, 102′, 102″ by a wire 101, 101′, 101″. In one embodiment, the device 100 includes three electrode arrays 102, 102′, 102″. It is noted, however, that any number of arrays 102 may be used without deviating from the scope of the invention. In an exemplary embodiment, each of the arrays 102 is 6-8 cm long, 2-7 mm or 3-7 mm thick, and 0.5-2 cm wide. A first one of the arrays 102 includes two contacts 103 and is connected directly to the implantable unit 104 by a first wire 101 having a first length. Second and third arrays 102′, 102″ include three contacts 103 and are connected directly to the implantable unit 104 by second and third wires 101′, 101″ having a second length greater than the first length. As shown in FIGS. 3-4, this configuration permits implantation of the device 100 such that first, second and third arrays 102, 102′, 102″ are separated from one another. It is noted, however, this embodiment is exemplary only and that any other combination of contacts 103 may be provided on the arrays 102 without deviating from the scope of the invention. The first, second and third arrays 102, 102′, 102″, respectively, may include 2-6 contacts 103 which may be separated from one another along a length of the arrays 102, 102′, 102″ by an inter-electrode distance of 1-6 cm. The contacts 103 may be formed of steel, platinum alloy or another conductive material as used for physiological monitoring. The inter-electrode distance is selected to increase sensitivity to ictal activity while minimizing noise. The first, second and third arrays 102, 102′, 102″, respectively, are reinforced with a flexible mesh or string of a high tensile material embedded therewithin alongside or above the contacts 103 and their associated electrical connections, the reinforcement increasing a strength of the arrays to permit long-term implantation thereof. Any of the arrays 102, 102′, 102″ may further include one or more pressure sensors 105 on a galeal surface (i.e., a surface located externally of the skull in an implanted configuration) to record pulse and provide a measure of heart rate than can be added to a seizure detection algorithm, as will be described in greater detail later on. It is noted that the placement of the sensor 105 in FIG. 2 is exemplary only and that any number and location of the sensor 105 may be selected without deviating from the scope of the invention. Any of the arrays may further comprise one or more biometric sensors 107 embedded thereon, the biometric sensors 107 capturing one or more of pulse, blood oxygenation data, motion, acceleration, galvanic resistance, temperature, electro-myographic data, pressure and rheologic changes.

In an operative configuration, both the electrode arrays 102, 102′, 102″ and attached implantable unit 104 are implanted subgallealy. This configuration permits the insertion of the arrays 102, 102′, 102″ and unit 104 through a single incision in a single outpatient procedure, which reduces risks that are generally encountered by implanting components at different locations in the body. Furthermore, the co-implantation reduces the risk of wire fracture due to the shorter wire length required. The exemplary device 100 produces a negligible amount of functional heat. Any heat produced is readily transferred or dissipated away from the inherent blood perfusion ability of the scalp. Nevertheless, the unit 104 is formed of an insulative material to prevent discharge of heat from the processor. The unit 104 comprises an analog-to-digital converter 108 to convert detected brain wave signals into digital signals at a predetermined sampling rate to adequately record relevant brain activity data. Any number and arrangement of amplifiers 110 and filters 112 are provided to enhance or modify the brain activity data for analysis and detection of specific information. A processor 106 runs a seizure detection algorithm on the brain activity data and identifies epileptic events based on this brain activity data. The processor 106 sends this data to a memory 116 where epileptic event data is stored. The epileptic event data may then be forwarded to a transceiver 114 as for transmission to a remote host (e.g., computer, mobile phone, etc.) via low-energy radio (telemetry) technology. In one embodiment, the processor 106 transmits only epileptic event data to the memory 115 and discards extraneous brain activity data. In an alternative embodiment, the processor 106 transmits epileptic event data wirelessly to a remote host without storage in an attached memory if the remote host is within a predetermined proximity to the patient. In yet another embodiment, the processor 106 saves the epileptic event data to the memory 116 only when the remote host is inaccessible (e.g., when the wearer is not within a transmission range to the remote host). In yet another embodiment, the processor 106 saves epileptic event data to the memory 116 and only transmits the event data to the remote host upon (1) receiving a transmission request from the host, (2) when memory usage reaches a predetermined level, (3) expiration of a predetermined time interval (e.g., daily, weekly, bi-weekly, monthly, etc.) or (4) when a critical number of epileptic events have occurred within a predetermined time period. The memory 116 may further comprise a database 118 containing information necessary for the analysis of brain activity data to detect epileptic events as well as threshold levels and other settings that may be referenced by the processor 106. Once transmitted (off loaded from device memory 116) the data is transferred to and stored locally in a smart device (smart phone), base station, or personal computer, or any other location including, for example, an internet cloud or a specific website. Information associated with the device 100 includes brain electrical data, data corresponding to time of day and circumstances of associated metrics, biometric data (e.g., pulse, body temperature, activity such as motion, etc.) and data captured from one or more pressure sensors 105 or other sensors provided on the arrays 102, 102′, 102″. Secondary physiologic measures such as heart rate either as a simple metric or as an analyzed time series can be recorded and added to a seizure detection algorithm. In some embodiments, a warning or alarm may be activated if a potentially dangerous nocturnal seizure is detected.

In accordance with an exemplary method according to the invention, the device 100 is inserted subgalleally using a minimally invasive surgical method, as those skilled in the art will understand, and implanted at a cranial vertex. Specifically, a blunt and tapered tip malleable dissector is passed into the subgaleal space by the surgeon to create a linear pocket into which the electrode arrays 102, 102′, 102″ are passed. Though the device 100 is typically implanted at the cranial vertex, other implantation locations on the head are also envisioned within the scope of the invention. The exemplary invention has identified the utility of the vertex position in recording seizures arising from and involving multiple brain locations, including the temporal lobes and does not require a priori knowledge of the lateralization of seizures for the individual. Furthermore, the vertex position also allows identification of electrographic markers of sleep stages which are typically maximal over the central head regions allowing a reviewer to easily assess the state during which detected seizures occurred. Finally, the location of the contacts 103 at the vertex, distant to the temporalis muscles reduces the contamination of the captured signal by artifacts produced by chewing or other movements of the jaw muscles. In situations where the precise location of the patient's seizure onsets are known, the implanted arrays 102, 102′, 102″ can be positioned over the region of the skull closest to this brain region, maximizing the sensitivity of the device to detect the seizure discharge. Furthermore, the exemplary implantation location reduces wound healing time and is cosmetically preferable to other implantation locations. In one embodiment, the implanted device 100 may extend along the axis of the sagittal suture. An orientation of each of the arrays 102, 102′, 102″ may be selected to correspond to the requirements of the patient. For example, the arrays 102, 102′, 102″ may be positioned to lie para-sagitally over the vertex of the scalp. After implantation, the device 100 may be optimized by causing the processor to continuously wirelessly transmit a subgaleal EEG signal to enable a surgeon or other user to fine-tune the location and orientation of the device 100 to achieve consistent and high quality brain electrical data or other biometric data. Once implanted, the device 100 monitors the subgaleal EEG continuously throughout the length of implantation via a low-power seizure detection process. In an exemplary embodiment, the device 100 is configured to operate on low-power to permit long-term implantation, wherein the battery powers the device for a period of at least 3 days, at least a week, at least 12 months, a period of up to 12 months, or a period greater than 12 months. Due to aging of the electrodes, changes in background activity, or tissue changes such as scarring of adjacent scalp tissue, the baseline signal characteristics such as noise level and signal-to-noise ratio may change for implantable devices during long-term use. The exemplary device 100 is configured to minimize and potentially eliminate the need for any intervention during the implantation period. To address any deterioration of the physiologic signals captured by the device 100, a technique may be implemented to identify and adjust to the changes in the baseline characteristics of the signal by manipulation of impedance or changes in electrode recording position or changes in the electrode sampling (i.e., electrode combination changes that can be programmed into the unit 104 as needed).

As shown in FIG. 5, a device 200 according to another embodiment of the invention is substantially similar to the device 100 except as noted below. Whereas the arrays 102, 102′, 102″ of the device 100 are each coupled directly to the implantable unit 104 via wires 101, 101′, 101″, arrays 202, 202′, 202″ of the device 200 are connected to the implantable unit 104 (not shown) by a branching connection. Specifically, wires 201, 201′, 201″ extend from the arrays 202, 202′, 202″ to a junction 210. A single wire 211 extends from the junction 210 to the unit 104. Each of the wires 201, 201′, 201″ has a different length so that corresponding arrays 202, 202′, 202″ are separated from one another in the implanted configuration. In one embodiment, the first wire 201 is 6 cm long and the second and third wires 201′, 201″ are 8 cm long. It is noted that these measurements are exemplary only and that any other measurements may be used without deviating from the scope of the invention.

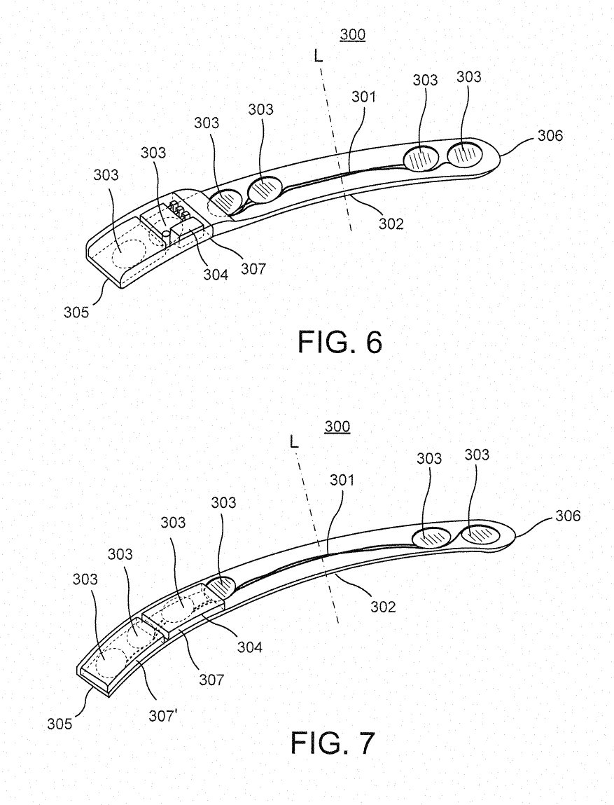

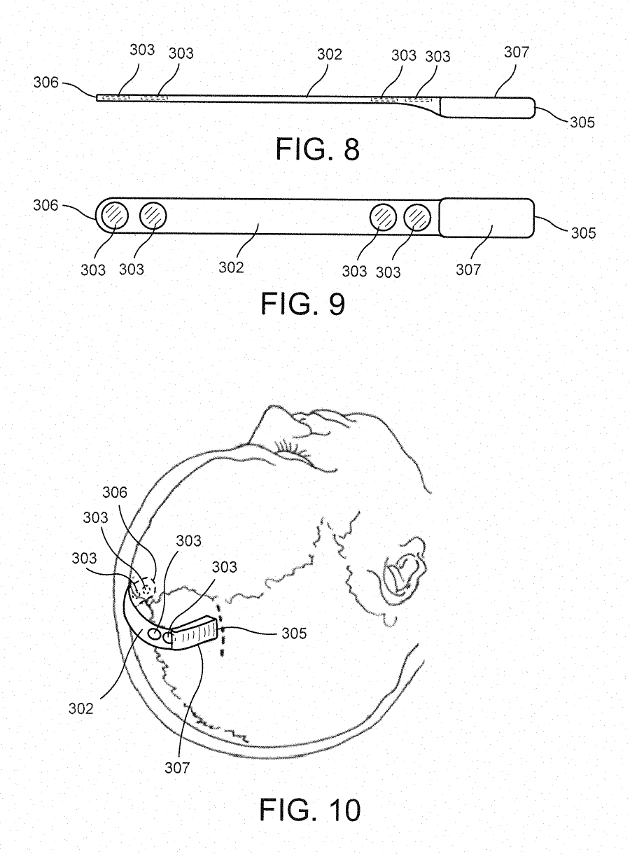

In another alternative embodiment shown in FIGS. 6-10, the exemplary device 300 may comprise a composite unit for detecting and analyzing brain activity data. The composite unit is preferably a single integrated device. It is believed that a single integrated device provides improved reliability as compared to multi-part devices. For example, the integrated device 300 requires the use of fewer lengthy leads, which are believed to be a cause of mechanical failure. In this particular embodiment, the exemplary device 300 may be a single integrated device that is in one piece and thereby reduces the number of lengthy leads, and thus, the risk of failure of the device attributable to mechanical failure of such lengthy leads. In addition, it is believed that a single integrated device allows a physician to more easily implant the device in a subgaleal region, and thus, provide for easier removal or revision of the device, shorter surgery time, and/or less invasive surgical procedure to place the device in an operative configuration. In addition, it is believed that the single integrated device/unitary device may be easy to implant as well as remove from the patient, because external wires are not necessary to connect multiple electrodes. Thus, separation of wires from surrounding tissue ingrowth or scar tissue would not be necessary and thereby easing subsequent removal of the device 300 from the patient.

The exemplary device 300 may comprise a low profile electrode strip 302, as show in FIGS. 6-10, which is sized and shaped for insertion into a subgaleal region of a patient. In one exemplary embodiment, the electrode strip 302 may have a length from about 4 cm to about 8 cm. The electrode strip 302 may be a single composite flexible electrode strip having a proximal end 305 and a distal end 306. The electrode strip 302 may be formed from any resiliently flexible material suitable for implantation. For example, the electrode strip 302 may be formed from or reinforced with a flexible mesh or string of a high tensile material.

In one embodiment, the electrode strip 302 may a central axis (L) bisecting the length of the electrode strip. The electrode strip 302 may comprise one or more electrode contacts 303 positioned proximal of the central axis, and one or more electrode contacts 303 distal to the central axis. In certain embodiments, the electrode strip 302 may include one or more electrode contacts 303 positioned proximal of the central axis, and one or more electrode contacts 303 distal to the central axis, wherein the electrode contacts 303 are configured to record brain activity data, e.g., EEG data of the patient's brain, by a single-channel recording. Preferably, the electrode strip 302 may comprise two to four contacts 303 proximal of the central axis, and two to four contacts distal of the central axis. In one particular embodiment, the electrode strip 302 may comprise one or more electrode contacts 303 positioned at or near each of the proximal end 305 and the distal end 306. Preferably, the electrode strip 302 may comprise two to four contacts 303 at or near each of the proximal end 305 and the distal end 306 of the electrode strip 302. The contacts 303 may be formed of steel, platinum alloy or other conductive material suitable for use for physiological monitoring. Preferably, the contacts 303 are formed from stainless steel. The contacts 303 on the electrode strip 302 may be configure to record brain activity data, e.g., EEG data of the patient's brain, by bipolar or referential recording.

In one particular embodiment, the electrode strip 302 may utilize only two electrode contacts 303 that record brain activity data in a single channel. The particular embodiment may further include additional electrode contacts 303 that remain inactive until a first pair of electrode contacts 303 fail. Specifically, the electrode strip 302 may include two pairs of electrode contacts 303: a first pair configured to record brain activity data in a single channel, and a second pair configured to remain inactive until the first pair fails and also configured to record brain activity data in a single channel. Alternatively, the electrode strip 302 may include more than two electrode contacts 303, but utilize only two electrode contacts 303 that record brain activity data in a single channel at any time. In particular, the exemplary device 300 may be configured to receive manual input from a user to select a pair of electrode contacts 303 on an electrode strip 302, or may be configured to determine which pair of electrode contacts 303 provide the best impedance and/or signal, e.g., selecting a combination of two contacts that provide the strongest signal out of all possible duo-contact combinations on the device 300.

The inter-electrode distance between contacts 303 at each of the proximal end 305 and the distal end 306 may be each independently selected to increase sensitivity to ictal activity while minimizing noise. The inter-electrode distance between contacts 303 may be selected to detect ictal activity with a signal to noise ratio of at least 1.1 to 1, 1.5 to 1, 2 to 1, 3 to 1, 5 to 1, or 10 to 1. More particularly, the inter-electrode distance may be selected to provide a signal to noise ratio from about 1.2 to 1 to about 100 to 1, from about 1.3 to 1 to about 75 to 1, or from about 1.5 to 1 to about 50 to 1. The signal to noise ratio discussed above may be for data covering an overall range of frequencies, or for a specific range of frequencies. In particular, the signal to noise ratio may be for a range of frequencies from about 0 to about 25 Hz, which is a range in which seizures are most prevalently detected. Moreover, the signal to noise ratio may be for a specific EEG frequency band, namely theta (e.g., between 4 Hz and 7 Hz) and alpha (e.g., between 8 Hz and 12 Hz), which are two frequency bands in which ictal activity is most often detected. In particular, the inter-electrode distance between contacts 303 may be from about 1 cm to about 10 cm, from about 3 cm to about 7 cm, from about 3 cm to about 5 cm, or from about 5 cm to about 6 cm. More particularly, the electrode strip 302 may include two contact 303 that are spaced apart at an inter-electrode distance of about 3 cm to about 7 cm, or from about 3 cm to about 5 cm. The electrode strip 302 may also include one or more sensors on a galeal surface to record pulse and provide a measure of heart rate. In other embodiments, the electrode strip 302 may also comprise one or more biometric sensors capturing one or more of pulse, blood oxygenation data, motion, acceleration, galvanic resistance, temperature, electromyographic data, pressure and rheologic changes.

The contacts 303 on the electrode strip may be connected to an implantable unit 304 by a wire 301. Each contact 303 may be independently connected via a wire 301 to the implantable unit. Alternatively, the contacts 303 may be connected to wires 301 forming a branching connection (not shown) that is connected to the implantable unit 304. The implantable unit 304 may be similar to the implantable unit 104 describe above except as noted below. The implantable unit 304 may include a processor for executing a seizure detection process or algorithm on brain activity data collected by the exemplary device, e.g., contacts 303, and identifying epileptic events based on this brain activity data. The processor may send the data to a memory where epileptic event may be stored. The implantable unit 304 may also include a transceiver for transmitting the epileptic event data to a remote host (e.g., computer, mobile phone, etc.) via low-energy radio (telemetry) technology. In some embodiments, the implantable unit 304 may be in communication with an external communication device where both the implantable unit 304 and the external device provide power to establish the communications. In particular, the implantable unit 304 may include a transceiver that utilizes a low-energy communications technology, wherein power requirements for such communications is provided predominantly, e.g., greater than 50%, by the external device. Additionally, the implantable unit 304 may comprise an analog-to-digital converter to convert detected brain wave signals into digital signals, preferably at a predetermined sampling rate to adequately record relevant brain activity data. The implantable unit 304 may also include any number and arrangement of amplifiers and filter to enhance or modify the brain activity data for analysis and detection of specific information. The implantable unit 304 may further comprise a battery for powering the components therein, e.g., processor, memory, transceiver, etc. In certain exemplary embodiments, the implantable unit 304 may comprise a flexible battery or a battery that can be separated to articulate with a curvature of a person's skull. In some exemplary embodiments the battery may be rechargeable wirelessly via an external source, such as, an external communications device.

The implantable unit 304 may be positioned at a proximal end 305 of the electrode 302 strip and may be enclosed within a housing 307. As shown in FIG. 6, the housing 307 is hermetically sealed around the implantable unit 304, which includes the battery. In some embodiments, the housing 307 may have a slightly curved shape that corresponds to a mean curvature of a human skull. It is believed that this curved housing shape more closely correlates to a curvature of a patient's skull and thus reduces the chance for skin erosion or breakdown of the exemplary device 300 caused by friction between the device and the patient, such as the patient's skin or skull. In certain embodiments, the housing 308 may be formed of an electrically conductive material. More particularly, the housing 308 may serve as a reference or ground for the exemplary device 300. In other embodiments, the housing 308 may be formed of an insulative material to prevent discharge of heat from the implantable unit 304. In some embodiments, the housing 308 may also include a mesh embedded therein. The mesh may reduce breakage or inelastic deformation or stretching of the housing 308 during implantation. In particular, the mesh may impart an improved tensile strength, and therefore, improve mechanical integrity of the housing 308 as compared to those formed with deformable materials that are not reinforced with a mesh therein.

In another embodiment, as shown in FIG. 7 the battery may be separately sealed within a second housing 307′ while the remainder of the implantable unit 304 is sealed with housing 307. The second housing 307′ may be similar to housing 307 as described above. In some embodiments, the second housing 307′ may be proximal of the housing 307. In other embodiments (not shown), the second housing 307′ may be distal to the housing 307. The exemplary embodiment shown in FIG. 7 allows for more flexibility at the proximal end 305 of the electrode strip 302. Therefore, the device 300 shown in FIG. 7 provides for more flexibility and can be more closely curved to correspond to the curvature over the patient's skull.

In an operative configuration the device 300 may be implanted subgalleally through a single incision at a cranial vertex of the patient. The exemplary device 300 allows for a minimally invasive implantation procedure, such as an incision having a size that is less than 5 cm, less than 3 cm, or less than 2 cm. In particular, a small incision having a size of about 1 cm may be sufficient for implantation of the exemplary device 300. More than one device may be used for each patient. An incision may be made for the implantation of each device 300. Alternatively, a single incision may be used for the implantation of a plurality of devices 300. It is believed that the subgaleal implantation of an EEG device, in particular a unitary device for recording EEG from the subgaleal region of a patient's brain, provides clinical benefits to the patient. Specifically, the implantation procedure of a subgaleal device 100, 200, 300 is less invasive as compared to implantation of intracranial electrode, which requires a craniotomy and drilling of burr holes to penetrate the skull bones so that the electrodes may make direct contact with the dura and/or brain tissue. Such intracranial operations are costly, requires a full operation that can last 3-6 hours, requires full anesthesia, involves increased risk of infection, and requires a long period of healing time. In contrast, subgaleal implantation of the exemplary device 100, 200, 300 may be performed in a much less invasive process that can be done in an outpatient process in about 10 minutes using local anesthesia or mild sedation. The healing time for subgaleal implantation is also significantly reduced as compared to implantation of intracranial electrodes, and also reduces the risk of infection to the implantation site.

The device 300 may be placed in any suitable position in a subgaleal region that is between the skull and the scalp of the patient. In certain embodiments, the device 300 may be implanted perpendicular to an anterior posterior head axis of the patient. In other embodiments, the device 300 may be implanted parallel to an anterior posterior head axis of the patient. In further embodiments, the device 300 may be implanted at an angle between 0 to 90° to the anterior posterior head axis of the patient. In another embodiment, the device 200 may be implanted at an angle between 0 to 180° to the anterior posterior head axis of the patient. As shown in FIG. 10, the device 300 may be placed across the cranial vertex, such that the device 300 positions its contacts 300 to cover both hemispheres of the patient's skull. For example, the proximal end 305 of the electrode strip 302 may lie in one hemisphere (e.g., right side) of the patient's skull while the distal end 306 lies on the other hemisphere (e.g., left side) of the patient's skull. Although not shown in FIG. 10, it is also contemplated that the proximal end 305 of the electrode strip 302 may lie on the left side of the patient's skull while the distal end 306 lies on the right side of the patient's skull. The device 300 may be positioned such that at least one contact 300 is positioned on each hemisphere (e.g., right side or left side) of the patient's skull. Preferably, at least two to four contacts 303 may be positioned on each hemisphere of the patient's skull.

In some embodiments, a single device 300 may be sufficient for recording EEG data and detection of epileptic events. In particular the device 300 may be positioned at a cranial vertex of the patient. More particularly, the device 300 may extend between a right hemisphere and a left hemisphere of the patient's skull. Alternatively, the device 300 may be positioned over a region of the skull closest to a portion of the brain to which the patient's seizure onsets were previously detected, or otherwise known, thereby maximizing the sensitivity of the single unitary device 300 to detect seizure discharge within the patient. In another alternative embodiment, more than one device 300 may be implanted in a subgaleal region between the skull and the scalp, and at or near the cranial vertex of the patient. In certain embodiments two or more devices 300 may be subgalleally implanted into the patient. For example, one device 300 may be implanted to cover one hemisphere of the patient's skull while a second device 300 is implanted to cover the other hemisphere of the patient's skull. More particularly, each device 300 may be positioned so as to broadly cover one hemisphere of the patient's skull.

In particular, the device 300 may be implanted subgalleally at a cranial vertex of the patient. As discussed above with respect to device 100, the utility of the vertex position in recording seizures arising from and involving multiple brain location, including the temporal lobes, and does not require a priori knowledge of the lateralization of seizures for the individual. Furthermore, the vertex position also allows identification of electrographic markers of sleep stages which are typically maximal over the central head regions allowing a reviewer to easily assess the state during which detected seizures occurred. In addition, the positioning of the device 300 may be at least 1 cm away from the temporalis muscles and thereby reducing the contamination of captured signal by artifacts produced by chewing or other movements of the jaw muscles. In some examples, the device 300 may be positioned over an insertion fascia of the temporalis muscles. In other examples, the device 300 may be positioned such that a longitudinal axis of the device 300 is substantially parallel to an axis of the sagittal suture of the patient. In other examples, the device 300 may be positioned such that a longitudinal axis of the device is substantially perpendicular to the axis of the sagittal suture of the patient.

The exemplary device 100, 200, 300 may be configured as a brain state monitor. In particular, the exemplary device 100, 200, 300 may be configured for subgaleal detection and recordation of brain physiological/electrophysiological data, e.g., EEG, and thereafter distinguishing one brain state from another based on the subgaleal EEG data. The exemplary device 100, 200, 300 permits the subgaleal detection of epileptic events, minimizing and/or eliminating false positives, while detecting all or nearly all seizure events. By contrast, no techniques for long-term monitoring currently exist. The exemplary device 100, 200, 300 provides a small, safe, fully implantable chronic SG extracranial EEG monitoring system, which offers a safer approach to the analysis and treatment of epilepsy. In addition to providing immediate and accurate objective data corresponding to epileptic events, the exemplary device 100, 200, 300 also reduces the time and number of patients needed for drug or device trials by half or more, thereby reducing research, development and approval costs for such drugs and/or devices. In addition to reducing time, the device 100, 200, 300 also objectifies the data, thereby providing a new unbiased measurement not presently available to seizure therapy or experimental drug trials. In addition to its use for seizure detection, the device 100, 200, 300 may also be used in various areas of research and clinical management in current medical domains and in those domains in development including, but not limited to, sleep, psychiatry, physiology, and many other domains where chronic (long-term) EEG detection and analysis has relevance, as well as having immediate important biometric related applications for medicine, military, aerospace, biomedical clinical research, etc.

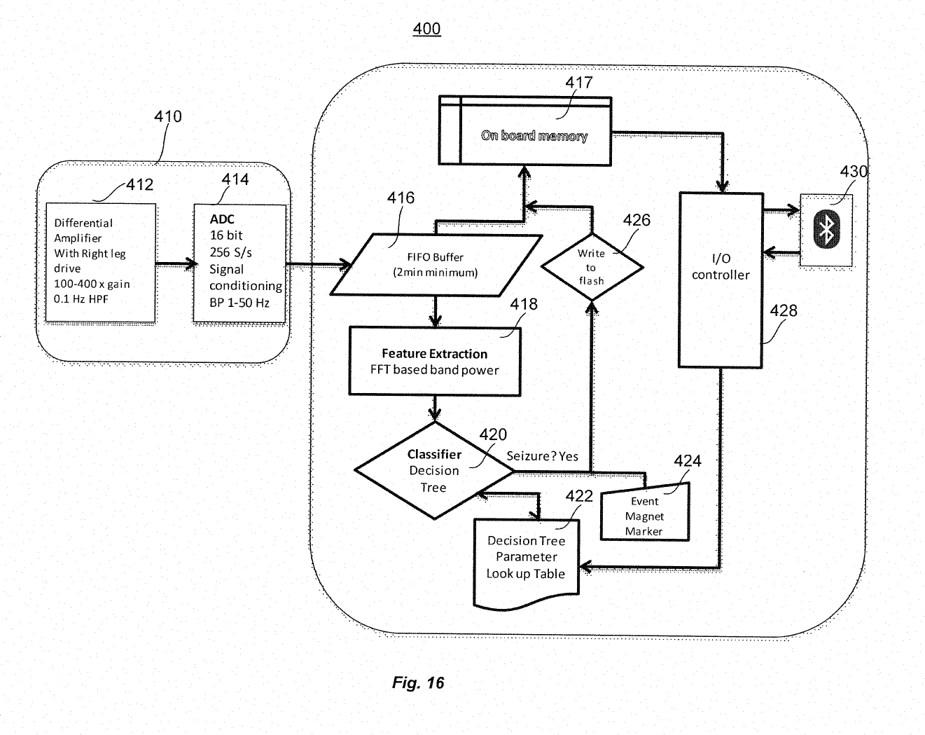

In one exemplary embodiment, the device 100, 200, 300 may be configured to record EEG data from a subgaleal region of the patient, and analyze the subgaleal EEG data for the occurrence of epileptic events. In particular, the device 100, 200, 300 may include a processor for executing a seizure detection algorithm on the brain activity data, e.g., subgaleal EEG data, and identify epileptic events based on this data. The processor may execute computer executable instructions stored on a memory or may send brain activity data to the memory when an epileptic event is detected. FIG. 16 shows a schematic diagram for a subgaleal EEG device 400 according to the present invention. The device 400 may include electrode contacts that are configured to detect brain activity/electrophysiological data and transmit the data to a signal processing device 410. The signal processing device 410 may include a digital differential amplifier 412 and an analog to digital converter 414. The digital differential amplifier 412 may be configured with a driven right leg circuit to reduce interference and may also be configured to amplify the gain of the brain activity/electrophysiological data by any suitable amount, such as, for example, by 100-400 times. The amplified data may then be subsequently filtered by the digital differential amplifier 412 with a high-pass filter (HPF) of 0.1 Hz. The amplified data is then transferred to the analog to digital converter (ADC) 414 where it may be converted to 16-bit digital data at a rate of 256 S/s. The ADC may further condition the signal with bandpass of 1-50 Hz.

As gain is increased bit depth becomes redundant as inefficient use of data density. For example, if one looks at slow activity such as sleep, one may want lower gains but appropriate resolution. In certain embodiments, the gain may be sacrificed for higher bit depth such that the signal processing device 410 may be an integrated unit such as a low-noise 8 channel, 24 bit analog front-end for biopotential measurements commercially available from Texas Instruments as TI ADS1299. This particular device is only exemplary and that it is contemplated that other suitable signal processing devices may also be used.

The digitized data may be transferred to a buffer, preferably a first-in-first-out (FIFO) buffer 416 for storing the data for any suitable time period, e.g., at least 2 mins, before further analysis. This buffered data is particularly useful as a baseline in comparison to data correlating to seizure activity. In particular, a pre-seizure baseline is often required for visual confirmation of seizure activity on EEG. By storing a short historical data buffer, the device can ensure an adequate recording of such baseline prior to activation of the seizure detector which may take 10-30 seconds to recognize a seizure. Furthermore, patients or caregivers may not be able to activate the device immediately at the onset of a seizure event. Moreover, keeping the short historical data buffer may also allow for subsequent retrieval of this data, without the need to immediately offload the data in real-time, before it is lost. This retrieval may be via manual, computer analytics, internal or external means and may include individual data or pooled data of many patients, and/or from individual or pooled seizure data of single patient. The buffered data may be automatically stored to a memory storage 417 within the device 400 or may be stored only upon detection of an epileptic event. The buffered data may be subsequently passed to a feature extraction module 418 for extracting a plurality of features from the digitized brain activity data. In one exemplary embodiment, the feature extraction module 418 may extract a plurality of features using a Fast Fourier transform (FFT) based on the band power of the subgaleal EEG data. In one particular embodiment, the subgaleal EEG data may be analyzed to obtain eight different features using a FFT. The extracted features are further analyzed using a classifier 420, which includes a decision tree for determining whether an epileptic event has been detected from the brain activity/electrophysiological data. The classifier 420 may encompass any suitable seizure detection algorithm, including a decision tree based on the plurality of extracted features.

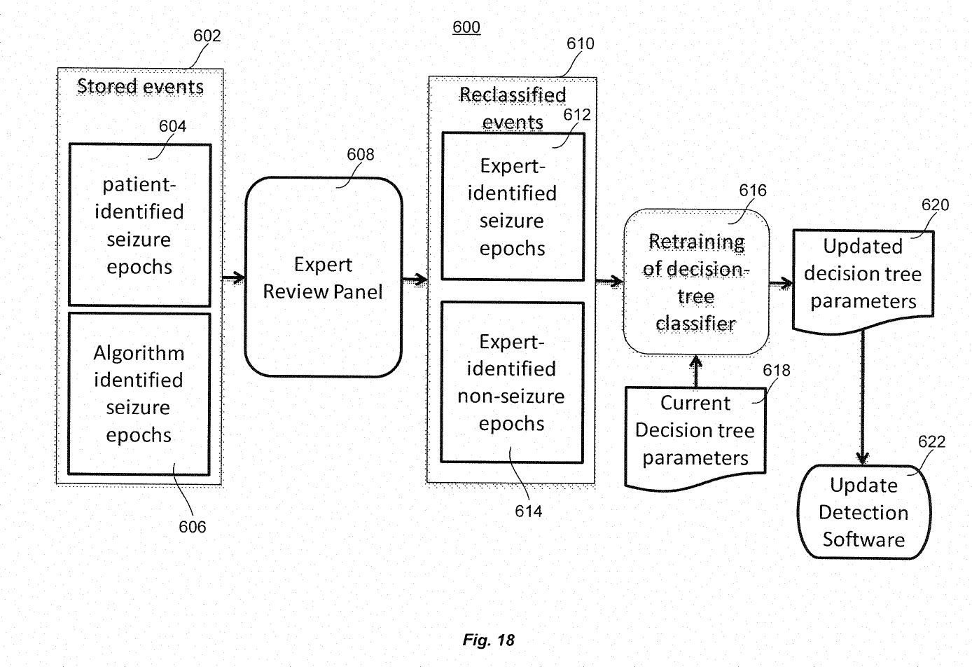

For example, each feature may be correlated to one or more parameters in a look up table 422. The parameters may be a set of predetermined values that are obtained from external source, e.g., manually inputted and transmitted to the processor, or may be a set of values generated from a database containing data and features recorded from numerous patients, including healthy patients as well as patients who suffer from epileptic seizures. In some embodiments, the decision tree and/or parameters may be continuously updated as new data is added to the database, as shown in FIG. 18 and discussed further below. If a seizure is detected by the classifier 420, an event magnet marker 424 may be triggered to indicate the detection of an epileptic event.

In some embodiments, a brain activity detector may include two stages. A first stage may comprise an analog threshold detector that triggers the activation of the second stage may correspond to the device 100, 200, 300, as described above, which is a more computationally intensive, e.g., utilizing the classifier 420 as described above. This two stage approach reduces power consumption by the on-board microprocessor. For example, the first stage may utilize a separate circuit to compute features from the EEG signal, such as integrated amplitude, root-mean square value across the broad band signal or within a narrow band. Such a circuit may be tuned to maximize sensitivity and activate the micropressor intermittently to perform the decision tree analysis to maximize specificity.

Upon detection of an epileptic event, the digitized brain activity data and/or its extracted features may also be written to a temporary memory storage, e.g., flash memory, as shown in step 426, which may subsequently write the data to a memory storage 417 within the device 400. The memory storage 417 provides information that may be outputted to an input-output (I/O) controller 428 that may be configured to transmits epileptic event data wirelessly to a remote host. The data may be wirelessly transmitted using any suitable wireless communications network 430, for example, Bluetooth, infrared, radio frequency, IEEE 802.1x, etc. The data may be transmitted using passive or active communication methodologies. In some embodiments, the digitized brain activity data and/or its extracted features may be collected simultaneously with other physiological data, such as, for example, altering skin conductance, sound, magnetic field, etc.

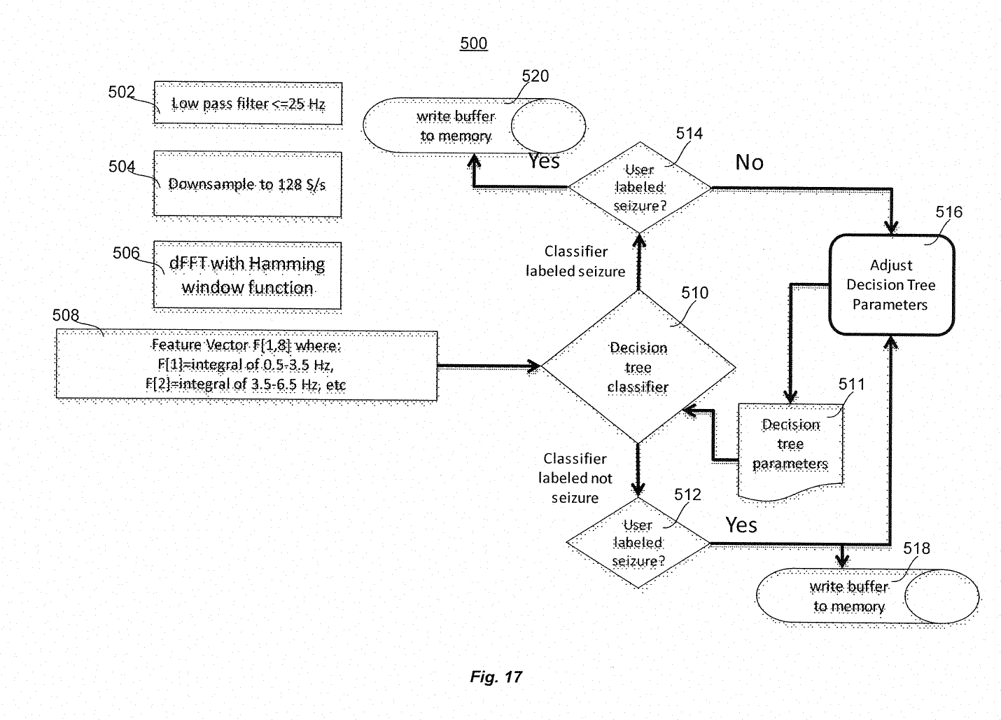

FIG. 17 shows an exemplary method 500 for detection of seizure using subgaleal EEG data. The brain activity data, e.g., EEG data, recorded from a subgaleal region of the patient may be subject to a low pass filter of less than 25 Hz (step 502) and down sampled to a rate of 128 S/s (step 504). It is noted that step 414, as discussed above, may be converted at a rate of 256 S/s, as which is a typical frequency at which clinical scalp EEG is recorded. However, in step 502, the brain activity data may be down sampled to a lower rate, e.g., 128 S/s. This down sampling improves the speed of computation for the exemplary method 500. However, higher sampling rates, e.g., 256 S/s, may preserve additional features of the EEG data may not be clear under a lower sampling rate, e.g., 128 S/s. The additional features of EEG data at a higher sampling rate may assist in visual review of the EEG data. It is noted that the brain activity data recorded from the subgaleal region of the patient may be analyzed, without use of an artifact reduction step, to determine whether an epileptic event is present. The filtered and down sampled subgaleal EEG data may then be digitized and analyzed using a FFT to extract a plurality of features based on the band power of the subgaleal EEG data (step 506). In particular, a dFFT process using a Hamming window function may be used to extract a plurality of features from the subgaleal EEG data. For example, the dFFT process may extract eight separate feature vectors from the subgaleal EEG data each having a band range as follows: (1) 0.5-3.5 Hz, (2) 3.5-6.5 Hz, (3) 6.5-9.5 Hz, (4) 9.5-12.5 Hz, (5) 12.5-15.5 Hz, (6), 15.5-18.5 Hz. (7) 18.5-21.5 Hz, and (8) 21.5-25 Hz. It is noted that the above number of feature vectors and band ranges are only exemplary and that any suitable number of feature vectors having different band ranges may be used. The extracted features are further analyzed using a decision tree classifier 510 for determining if an epileptic event has been detected from the brain activity/electrophysiological data (step 514) or not (step 512). The decision tree classifier 510 may utilize a set of decision tree parameters 110 which may be a set of predetermined values that are obtained from external source, e.g., manually inputted and transmitted to the processor, or may be a set of values generated from a database containing data and features recorded from numerous patients, including healthy patients as well as patients who suffer from epileptic seizures. In some embodiments, the parameters may be continuously updated as shown in step 516 and in FIG. 18 and discussed further below. If an epileptic event has been detected, the patient may be identified as having suffered from a seizure (step 514). The data may subsequently be used to update and/or adjust the decision tree parameters 511 via a supervised learning module (step 516). In addition, the brain activity data and/or its extracted features may be written to a buffer memory, e.g., flash memory, as shown in steps 518 and 520, which may subsequently transmit the data to a memory storage, to an input-output device, or via a wireless network to a remote host.

As discussed with reference to FIGS. 16 and 17 above, the brain activity data, e.g., EEG data, recorded from a subgaleal may be analyzed using a classifier and a decision tree having a plurality of parameters. FIG. 18 shows an exemplary embodiment of a decision tree 600, which may include a database 602 for containing data and features recorded from numerous patients, including healthy patients as well as patients who suffer from epileptic seizures. The database 602 may include patient data along with patient-identified seizure epochs 604, which are occurrences of seizure activity as reported by the patient, and algorithm identified seizure epochs 606, which are seizure activity identified by any suitable seizure detection algorithm, as already discussed above. The patient data along with the patient-identified seizure epochs 604 and the algorithm identified seizure epochs 606 may be outputted for review by an expert review panel, such as neurologist and researchers, and adjustments may be inputted to the database via an input-output device 608. A reclassified database 610 may be generated based on the input from the expert review panel to include expert-identified seizure epochs 612 and expert-identified non-seizure epochs. In some embodiments the reclassified database 610 may be separate from the database 602. In other embodiments, the reclassified database 610 may replace the database 602. The reclassified database 610 may be by a module 616 for retraining the decision tree classifier, where the current decision tree parameters 618 are inputted to the module 616 and a set of updated decision tree parameters 620 are outputted by the module 616 based on the reclassified database 610. The updated decision tree parameters 620 may be used to generate an updated process 622 for detecting the occurrence of seizure in subgaleal brain activity/EEG data. These adjustments to the decision tree 600, as discussed above, may be used useful in providing adjustments for improved detection of a seizure in a patient, or may be used to provide adjustments for improved detection of a particular type of seizure in the patient.

The exemplary device 100, 200, 300 may also be configured for subgaleal detection of epileptic events at a low power recording and a low sampling rate, as compared to conventional devices. Traditional portable (ambulatory) EEG devices record 22+ channels at 512 samples per second, which typically has a limited battery life of about 72-96 hours. The exemplary device 100, 200, 300 may be configured to utilize only a single channel at a time and/or at lower sampling rates, which reduces the electrical needs of the device. Furthermore, the device 100, 200, 300 may also be configured to require less onboard storage as compared to traditional EEG devices, for example, the device may be configured to only store epochs of interests as identified by exemplary method 600, which will further enable prolonged monitoring of the patient using a such a portable device implanted in the patient.

The exemplary device 100, 200, 300 may be used in combination with a therapeutic device directed to the treatment of seizures including, but not limited to, antiepileptic medications, implantable stimulators (e.g., electrical stimulators) and implantable drug delivery systems. Specifically, the device 100, 200, 300 may aid in monitoring the administration of medication to a patient by detecting breakthrough seizures and allowing for the timely collection of AED serum levels, as those skilled in the art will understand. The device 100, 200, 300 may also be used in conjunction with a device for monitoring recurring seizures, as those skilled in the art will understand. The device 100, 200, 300 may be programmed to distinguish between partial and generalized seizures in patients with partial epilepsy and also to distinguish between epileptic and non-epileptic seizures. The device 100, 200, 300 may quantify spike wave frequency and duration in patients with generalized epilepsies (e.g., absence epilepsy, JME, JAE, idiopathic GTCs, etc.). Furthermore, the device 100, 200, 300 may be used to rule out the diagnosis of epilepsy in a patient with an explained fall or loss of consciousness. The device 100, 200, 300 may be configured to provide any suitable external visual, auditory and/or mechanical notification to the patient, family, care provider, and/or other designated person or entity via communications to another device via any suitable communication system. For example, the device 100, 200, 300 may provide any of the above information to any suitable communication device, such as, but not limited to a cellular phone, a smart phone, a computer, a mechanical device, an automobile, a transportation vehicle, heavy machinery, a house-hold appliance, a robotic assistant or machine, etc.

The device 100, 200, 300 may also identify a slowing of the EEG that occurs with cerebral ischemia, elevated intracranial pressure, or hypoperfusion (cardiac, non-cardiogenic syncope). The device 100, 200, 300 may record oscillatory EEG activity associated with normal cerebral functions such as movement, vision, and sensation. The resultant data may be employed in the afferent arm of a therapeutic feedback system (e.g., neuro-prosthesis). The device 100, 200, 300 may also record oscillatory EEG activity associated with abnormal cerebral functions such as tremor, tics, attentional issues, or behavioral abnormalities. It is noted that the device 100, 200, 300 may record not just abnormal states, but normal states as well, such as, for example, wakefulness, attention, vigilance, etc. In addition, the device 100, 200, 300 may be configured to detect the effect of drugs upon the EEG of the patient. The resultant data may be employed in the afferent arm of a therapeutic feedback system using pharmacological or electromagnetic stimulation. Still further, the device 100, 200, 300 may monitor depth of coma/sedation in patients undergoing long-term sedation in critical care settings. The device 100, 200, 300 may be used for the long-term monitoring of physiological brain rhythms including but not limited to sleep-related oscillations and transients for the purpose of research in human physiology, sleep and memory.

There are many modifications of the present invention which will be apparent to those skilled in the art without departing from the teaching of the present invention. For example, although the exemplary embodiments have been described with respect to the detection of seizures, the exemplary devices 100, 200, 300 may be used for the detection of other conditions (e.g., mood disorders such as depression, Parkinson's Disease, schizophrenia, sleep disorders, autism, etc.) without deviating from the scope of the invention. The embodiments disclosed herein are for illustrative purposes only and are not intended to describe the bounds of the present invention, which is to be limited only by the scope of the claims appended hereto.

### EXAMPLE I

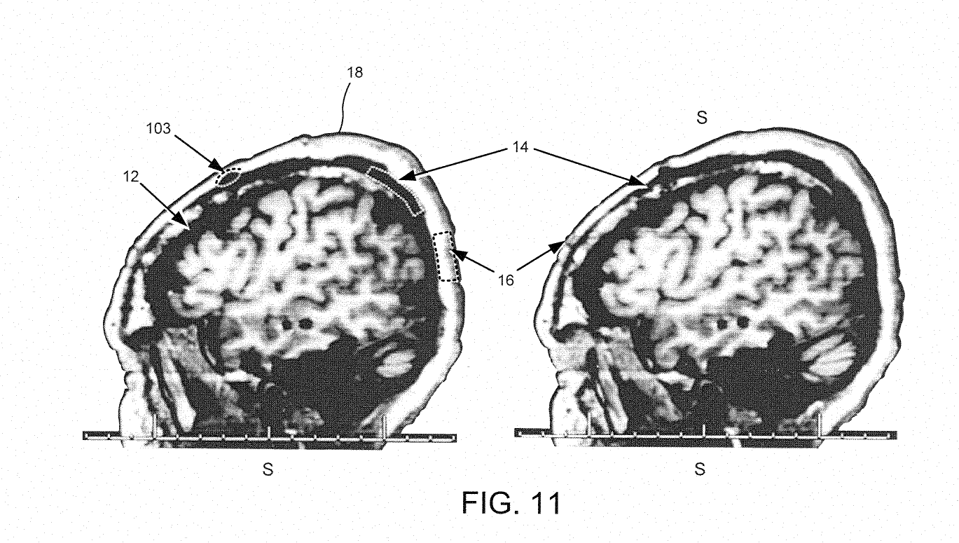

The exemplary device 100 may be implanted in a subgaleal region of a patient, near the brain, as shown in FIG. 11. As can be seen, at least one contact 103 (e.g., an electrode for recording electrophysiological data, particularly EEG data) of the device may be implanted in a subgaleal region that is between the skull 14 and the scalp 16 of the patient near the brain 12 of the patient. Specifically, the at least one contact 103 or electrode arrays 102, 102′, 102″ of the device 100 may be position perpendicular to a cranial vertex 18 of the patient. More particularly, the at least one contact 103 or electrode arrays 102, 102′, 102″ of the device 100 may lie across a mid-length of the patient's skull 14. It is contemplated that the exemplary device 200 may be alternatively implanted in the subgaleal region of the patient in the same manner described above and as shown in FIG. 11.

The device 100, 200 may be used to record EEG data from the patient extracranially without drilling through the patient's cranium for placement of any portion of the device 100, 200. The subgaleal device 100 provides a less invasive method as compared to electrodes implanted directly under the dura mater that is also capable of detecting changes in the patient's EEG data to identify seizures within seconds of the start of the seizure episode. In this example, data recorded by the subgaleal device 100, 200 is compared to EEG data recorded by electrodes implanted directly onto the dura mater.

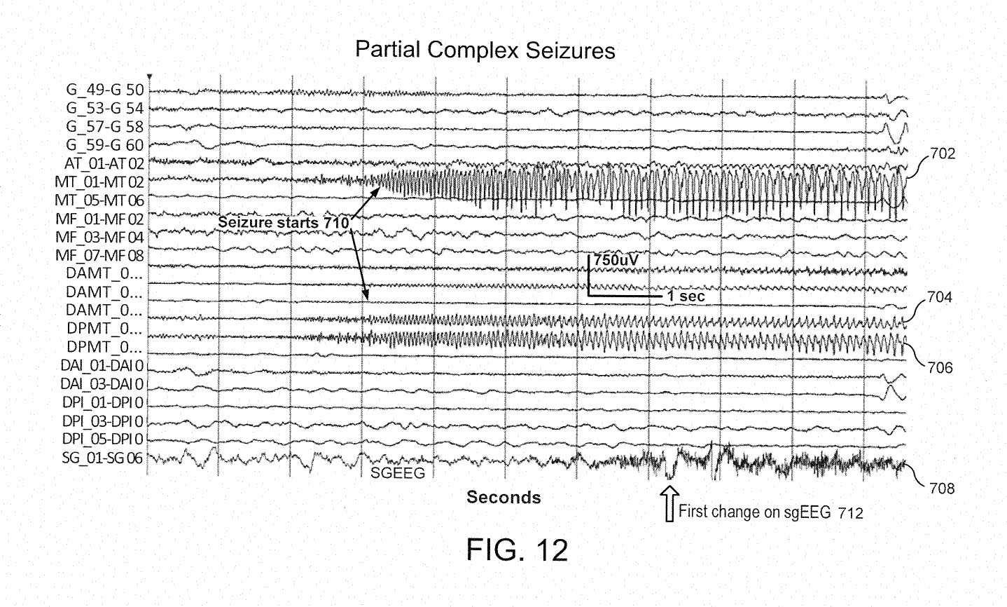

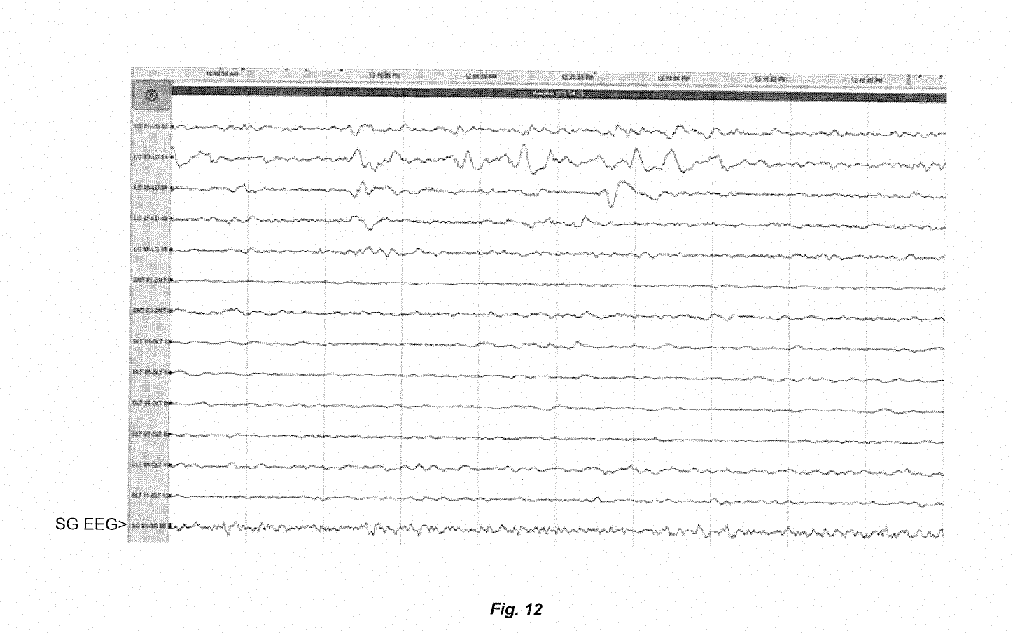

FIG. 12 shows the EEG data 702, 704, 706 recorded over time in a patient suffering from refractory partial complex seizures by electrodes implanted directly under the dura mater as well as EEG data 708 recorded by an exemplary subgaleal device 100, 200 of the present invention. In a particular embodiment, the at least one contact 103 or electrode arrays 102, 102′, 102″ of the device 100 may be position acrossed the vertex. Although this particular example refers to partial complex seizure, it is understood that the subgaleal device 100, 200 of the present invention may be used to detect any number of neurological disorders and other types of seizures. As shown at reference number 710, an excessive level of neurological activity may be record in EEG data 702, 704, 706 recorded by the electrodes implanted into the dura mater and signifies the onset of a seizure. The subgaleal device 100, 200 of the present invention may also be used to detect the onset of the seizure. As can be seen in FIG. 12, the EEG data 708 recorded by the subgaleal device 100, 200 may detect a deviation 712 from baseline signal characteristics within seconds, e.g., less than 5 seconds, of the onset of the seizure as recorded by the electrodes implanted into the dura mater. The EEG data 708 shown in FIG. 12 demonstrates that the subgaleal device 100, 200 may detect the onset of the seizure within approximately 3 seconds. The EEG data 708 recorded by the subgaleal device 100, 200 may be processed using any suitable seizure detection algorithm or may be visually examined by a physician to identify a seizure. The seizure detection algorithm may be executed by a processor 106 or may be part of a remote processing arrangement that receives the EEG data 708 recorded by the subgaleal device 100, 200. In certain embodiments, the seizure detection algorithm analyzes the EEG data 708 in real-time. Further, upon detection of a seizure, the subgaleal device 100, 200 may trigger an anti-seizure and/or anti-epileptic treatment. In particular, the subgaleal device 100, 200 may trigger an anti-seizure and/or anti-epileptic treatment immediately upon detection of the onset of a seizure. For example, the anti-seizure and/or anti-epileptic treatment may be administered in less than 5 seconds, within 3 seconds of the onset of the seizure.

### EXAMPLE II

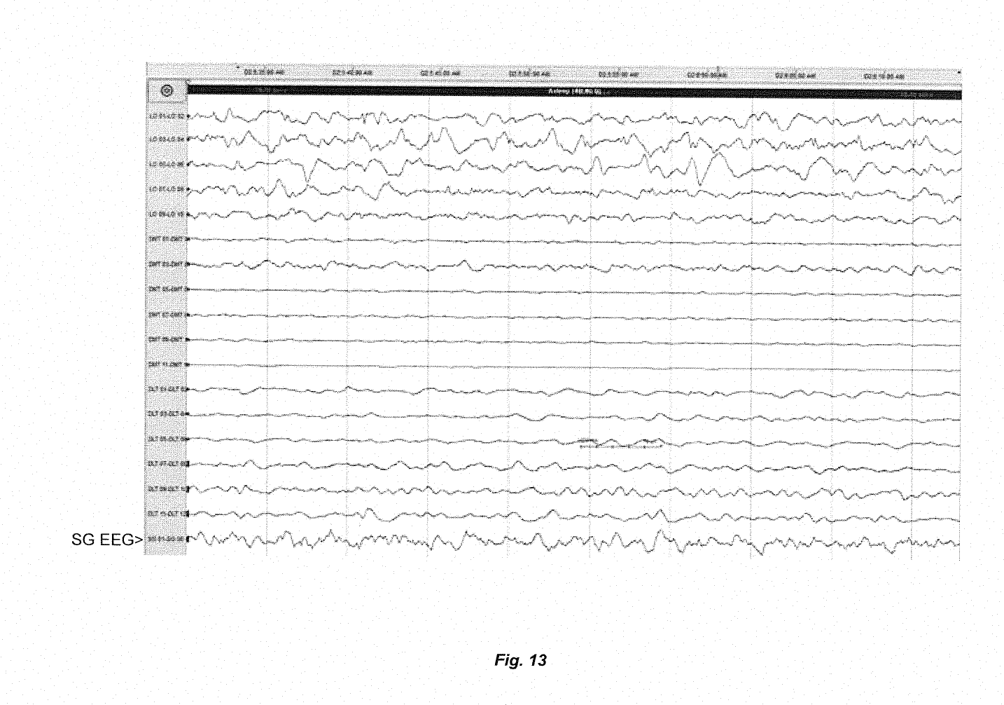

The exemplary device 100, 200, 300 may also be used to record EEG data from the patient that allows for identification of sleep stages (e.g., awake and sleep states). In particular the exemplary unitary device 300 as show in FIGS. 6-10 may detect sleep stages of a patient from a single device implanted at a single location, preferably at the cranial vertex. In Example II, the exemplary device 300 may be used to conduct a pre-surgical evaluation of a patient to record EEG data and determine electrophysiological signals for awake and sleep states of the patient. FIG. 13 shows the EEG data recorded over time in an awake patient who is being evaluated for epilepsy surgery by intracranial electrodes implanted directly onto the dura mater as well as EEG data recorded by an exemplary subgaleal device 300 of the present invention. A patient who is being considered for epilepsy surgery may be in one of two categories: (1) if their seizures are not their seizures are not adequately controlled by medications (e.g., failed to be treated by two or more for epilepsy); or (2) it is suspected that their seizures arise from a single focus. For these types of patients, epilepsy surgery, if possible, offers these patients the best chance of lasting seizure freedom.

In the data shown in FIG. 13, the top 13 data lines show EEG data obtained from intracranial depth and subdural electrodes, whereas the last data line, which is also labeled as “SG EEG,” shows EEG data obtained using electrodes that are implanted into the subgaleal space of the patient, such as the exemplary device 300. As can be seen in FIG. 13, it is difficult to determine a sleep stage using the intracranial EEG data of the top 13 data lines. This is because of focal cortical abnormality that is detected in intracranial EEG data that results in a continuous sharp and slow activity, regardless of the sleep stage, that interferes with brain activity that would be indicative of a sleep state or an awake state. However, this focal cortical abnormality is not recorded in EEG data recorded using a subgaleal device, e.g., exemplary device 100, 200, 300. Rather, as shown in the last data line of FIG. 13, the EEG data shows typical waking background frequencies.

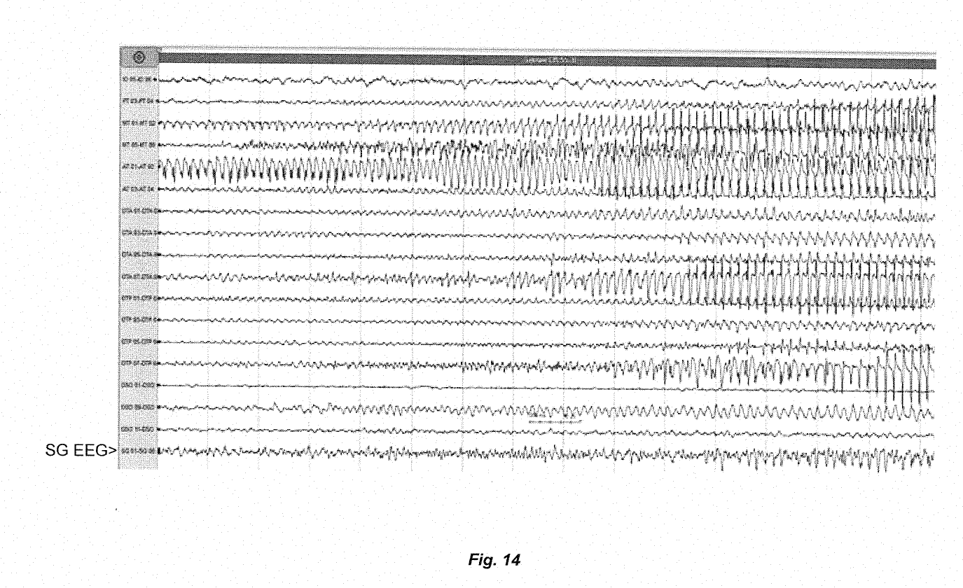

FIG. 14 shows EEG data recorded over time in an asleep patient who is being evaluated for epilepsy surgery by intracranial electrodes implanted directly under the dura mater as well as EEG data recorded by an exemplary unitary subgaleal device 300 of the present invention. In the data shown in FIG. 14, the top 13 data lines show EEG data obtained from intracranial depth and subdural electrodes, whereas the last data line, which is also labeled as “SG EEG,” shows EEG data obtained using electrodes that are implanted into the subgaleal space of the patient, such as the exemplary device 300. As can be seen in FIG. 14, it is difficult to determine a sleep stage using the intracranial EEG data of the top 13 data lines. However, as shown in the last data line of FIG. 14, EEG data recorded using a subgaleal device, e.g., exemplary device 300, shows an EEG pattern that is consistent with slow wave sleep. As demonstrated in FIG. 14, intracranial EEG provides a limited focus, whereas SG EEG data provides the ability to distinguish clinically important data from less important information that do not require any action. Although SG EEG does not necessarily provide better detection of seizure as compared to intracranial EEG—that is, it is different in some circumstances, yielding different data, specifically yielding data only for compromised patients in functional states, SG EEG is believed to provided better detection of functional state change characterization and/or identification as compared to intracranial EEG.

### EXAMPLE III

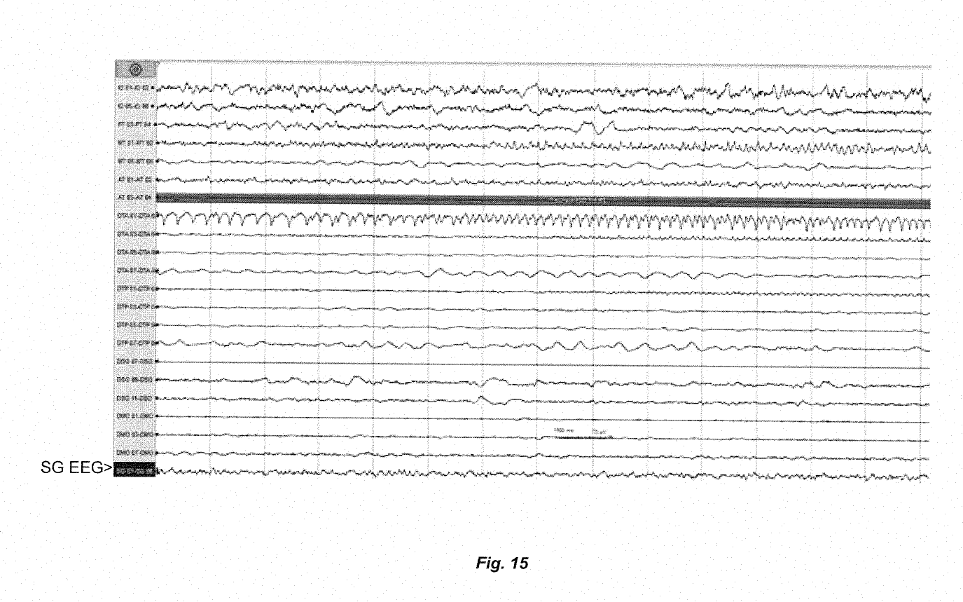

In Example III, the exemplary device 100, 200, 300 may be used to record EEG data from the patient and detection of epileptic activity. The exemplary device 300 may be used to conduct a pre-surgical evaluation of a patient to record EEG data and determine ictal activity of the patient. FIG. 15 shows the EEG data recorded over time in a patient who is being evaluated for epilepsy surgery by intracranial electrodes implanted directly onto the dura mater as well as EEG data recorded by an exemplary subgaleal device 300 of the present invention. In the data shown in FIG. 15, the top 17 data lines show EEG data obtained from intracranial depth and subdural electrodes, whereas the last data line, which is also labeled as “SG EEG,” shows EEG data obtained using electrodes that are implanted into the subgaleal space of the patient, such as the exemplary device 300, at the vertex on the right side. As can be seen in FIG. 15, complex partial seizure from the right temporal lobe may be characterized by behavioral arrest. The ictal rhythm of a complex partial seizure in the right temporal lobe may be observed in the top 17 data lines show EEG data obtained from intracranial depth and subdural electrodes, as well as in EEG data recorded using a subgaleal device, e.g., exemplary device 100, 200, 300.

FIG. 16 shows EEG data recorded over time in a patient who is being evaluated for epilepsy surgery by intracranial electrodes implanted directly onto the dura mater as well as EEG data recorded by an exemplary subgaleal device 300 of the present invention. Similar to FIG. 15, the top 17 data lines show EEG data obtained from intracranial depth and subdural electrodes, whereas the last data line, which is also labeled as “SG EEG,” shows EEG data obtained using electrodes that are implanted into the subgaleal space of the patient, such as the exemplary device 300. As can be seen in FIG. 16, the patient experienced a subclinical seizure originating in the right temporal lobe without any behavior changes—in this particular patient, the patient continued to use his cellular phone normally. The ictal activity is seen in the intracranial EEG data show in the top 17 data lines of FIG. 16. However, EEG data recorded using a subgaleal device, e.g., exemplary device 100, 200, 300, did not show a change in EEG that corresponds to ictal behavior. Therefore, the combination of the intracranial and subgaleal data show in FIG. 16 suggests that there is limited spread of the clinically silent seizure event.

### EXAMPLE IV

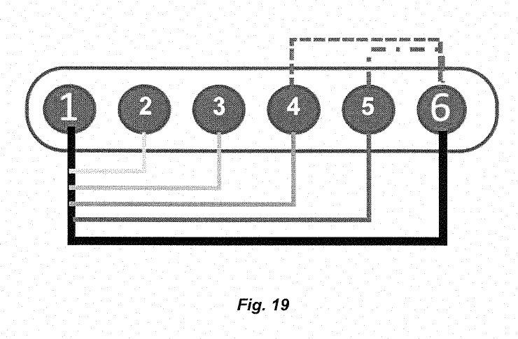

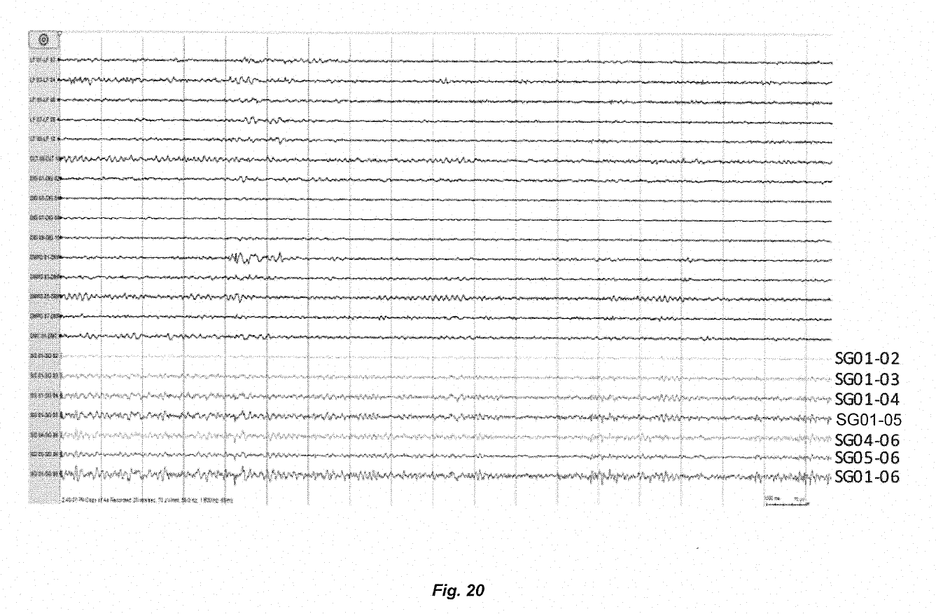

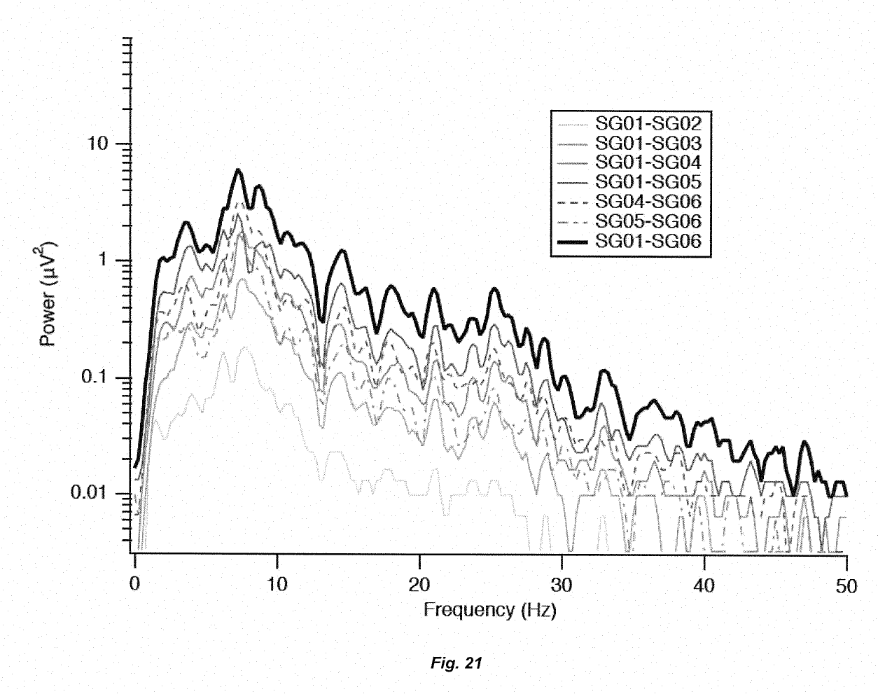

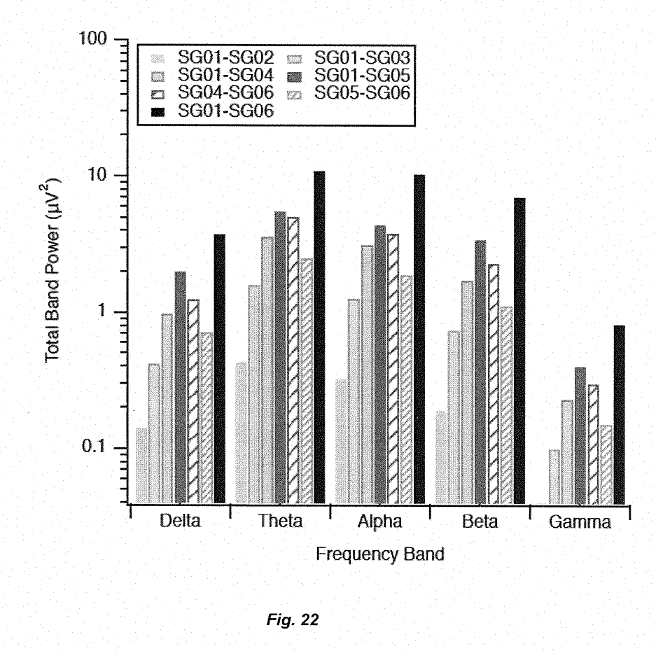

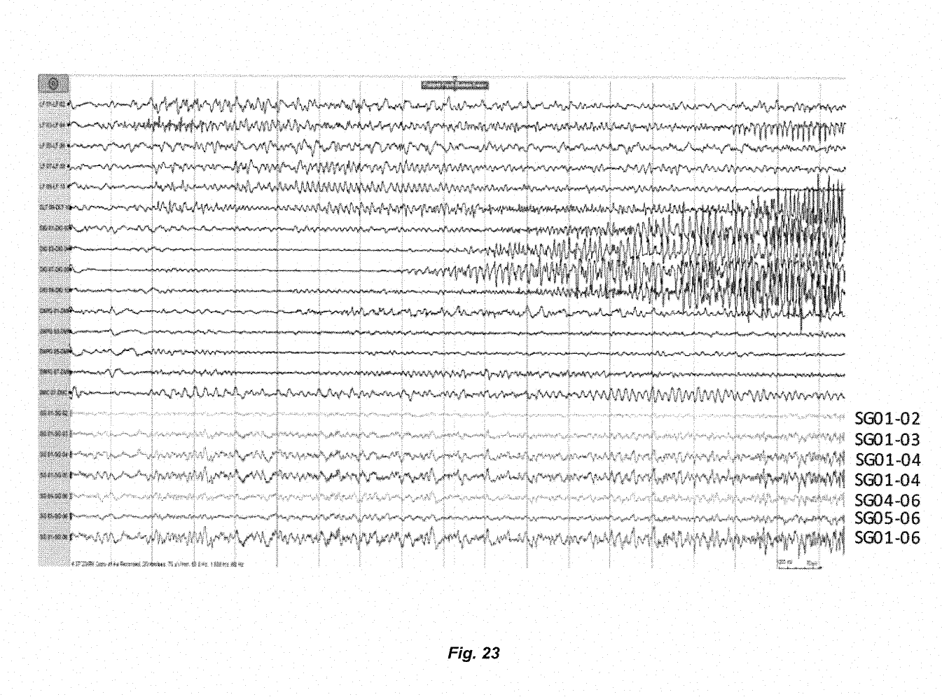

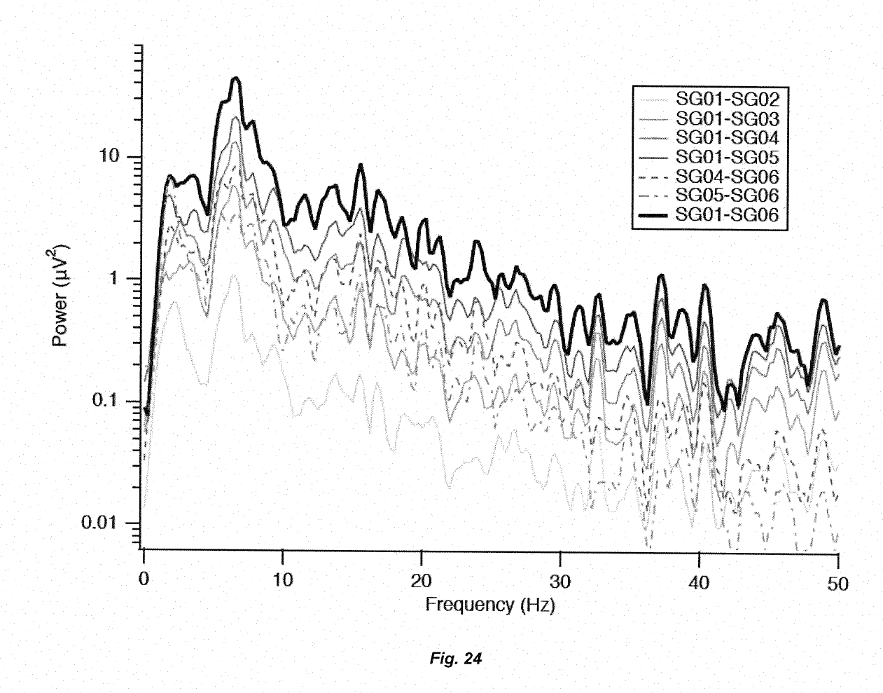

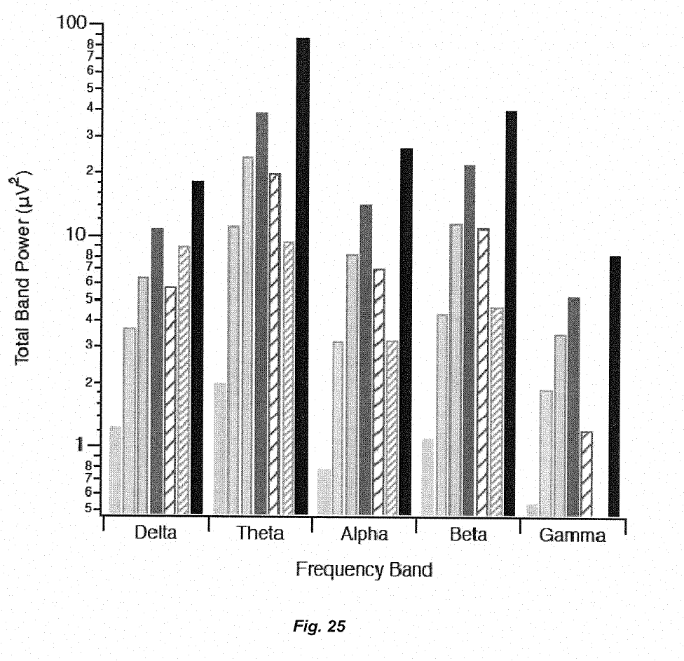

Example IV provides an exemplary embodiment of a single integrated device 300 having six different contacts 300, as shown in FIG. 19. However, only a pair of contacts 300 may be needed to record brain activity/electrophysiological data in a single channel from a patient. Data recorded between different pairs of electrodes 300, demonstrating the signal to noise ratio at different inter-electrode distances are shown below. The exemplary device shown in FIG. 19 includes 6 different circular contacts, each contacts being spaced apart about 1 cm from one center of a contact to another center of an adjacent contact. A first electrode is labelled with the number “1” and sequentially numbered to “6.” FIG. 20 shows the EEG data recorded over time in a patient during inter-ictal periods during sleep by intracranial electrodes implanted directly onto the dura mater as well as EEG data recorded by an exemplary subgaleal device 300 of the present invention. In contrast, FIG. 23 shows the EEG data recorded over time in a patient during a period including a complex partial seizure by intracranial electrodes implanted directly onto the dura mater as well as EEG data recorded by an exemplary subgaleal device 300 of the present invention. In both FIGS. 20 and 23, the top 15 data lines show EEG data obtained from intracranial depth and subdural electrodes. The data lines labeled with the prefix “SG” correspond shows EEG data obtained using the electrode array of FIG. 19, which is implanted into the subgaleal space of the patient. The lines labeled “SG01-02” correspond to single channel subgaleal EEG data recorded using contact 1 and 2, which corresponds to an inter-electrode distance of lcm. The lines labeled “SG01-03” correspond to single channel subgaleal EEG data recorded using contact 1 and 3, which corresponds to an inter-electrode distance of 2 cm. The lines labeled “SG01-04” correspond to single channel subgaleal EEG data recorded using contact 1 and 4, which corresponds to an inter-electrode distance of 3 cm. The lines labeled “SG01-5” correspond to single channel subgaleal EEG data recorded using contact 1 and 5, which corresponds to an inter-electrode distance of 4 cm. The lines labeled “SG04-06” correspond to single channel subgaleal EEG data recorded using contact 4 and 6, which corresponds to an inter-electrode distance of 2 cm. The lines labeled “SG05-06” correspond to single channel subgaleal EEG data recorded using contact 5 and 6, which corresponds to an inter-electrode distance of 1 cm. The lines labeled “SG01-06” correspond to single channel subgaleal EEG data recorded using contact 1 and 6, which corresponds to an inter-electrode distance of 5 cm. The subgaleal EEG data of FIG. 20 is further analyzed to provide a power spectral density plot in FIG. 21, and a band power density plot in FIG. 22. Similarly, the subgaleal EEG data of FIG. 20 is further analyzed to provide a power spectral density plot in FIG. 24, and a band power density plot in FIG. 25. Any suitable analytical methods may be used to obtain these power spectral density and band power density plot. In this particular example, the plots were obtained using the Natus NicOne EEG software.

The power spectral density plots of FIGS. 21 and 24 demonstrates a frequency range from 0 to 50 Hz the EEG power amplitude (in μV²) recorded by each pair of subgaleal EEG contacts from the electrode array shown in FIG. 19. The EEG power amplitude recorded by each pair of subgaleal EEG contacts are also analyzed based on different ranges of EEG frequency bands: delta (e.g., <4 Hz), theta (e.g., between 4 Hz and 7 Hz), alpha (e.g., between 8 Hz and 15 Hz), beta (e.g., between 16 Hz and 32 Hz) and gamma (e.g., >32 Hz).

As can be seen in FIGS. 20-25, in both the ictal and interictal recordings, signal amplitude and power for a broad range of frequencies was highest with an inter-electrode distance of 5 cm. Moreover, as shown in FIGS. 22 and 25, the amplitude of the ictal signals are particularly pronounced for the data labeled “SG01-05,” which corresponds to an inter-electrode distance of 4 cm, and the data labeled “SG01-06,” which corresponds to an inter-electrode distance of 5 cm. This data demonstrate that these particular inter-electrode distances provide improved signal to noise ratios such that an epileptic event may be more clearly observed. The improved signal to noise ratios allow for improved resolution of changes in EEG signal at multiple frequencies that can be used to identify changes in brain state including identification of epileptic seizures.

The exemplary embodiments described and claimed herein is not to be limited in scope by the specific embodiments herein disclosed since these embodiments are intended as illustrations. Any equivalent embodiments are intended to be within the scope of this application. Indeed, various modifications in addition to those shown and described herein will become apparent to those skilled in the art from the foregoing description. Such modifications are also intended to fall within the scope of the appended claims. All publications cited herein are incorporated by reference in their entirety.

What is claimed is:

1. A unitary implantable device comprising:
an elongated implantable body configured for implantation at or near a cranial vertex in a subgaleal extracranial space of a patient;
a first and a second electrode contacts separated from one another by a distance selected to form a single channel for detection of brain electrical activity;
a processor analyzing the detected brain electrical activity to determine whether a change in brain state has occurred and generating brain state data based on this determination; and
a transceiver controlled by the processor to wirelessly transmit epileptic event data to and from a remote computer.

2. The device of claim 1, wherein the processor is configured to continuously analyze the brain electrical activity.

3. The device of claim 1, wherein the change in brain state is an epileptic event, and the brain state data comprises epileptic event data.

4. The device of claim 1, wherein the implantable body includes a central axis bisecting the length of the implantable body, the first electrode contact being positioned proximal of the central axis and the second electrode contact being positioned distal of the central axis.

5. The device of claim 3, wherein the first and second electrode contacts are separated by a distance selected to form a single channel for detection of ictal activity with a signal to noise ratio of at least 1.1 to 1.

6. The device of claim 1, wherein the first and second electrode contacts are separated by a distance from about 1 cm to about 10 cm.

7. The device of claim 1, further comprising a memory coupled to the processor, the memory configured to store brain state data.

8. The device of claim 1, wherein the implantable body further comprises a battery.

9. The device of claim 1, wherein the device is configured for implantation in vivo for a period of at least 3 days

10. The device of claim 1, wherein the elongated implantable body comprises a housing hermetically sealed around the device.

11. The device of claim 1, wherein the implantable body includes a reinforced portion.

12. The device of claim 11, wherein the implantable body has a curved shape corresponding to a mean curvature of a human skull.

13. A method for capturing brain wave data, comprising:
inserting, using a minimally invasive surgical technique, an implantable body into a subgaleal extracranial position at or near a cranial vertex of a patient, the implantable body positioned along a cranial surface at least 1 cm away from the temporalis muscles of the patient, and so that first and second electrode contacts of a first electrode array of the implantable body face the cranium;
detecting brain electrical activity via a single channel formed by the first and second electrode contacts;
monitoring and analyzing, via a processor, the brain electrical activity to detect epileptic events; and
transmitting epileptic event data corresponding to a detected epileptic event to one of a remote computer, local computer, local base station, cellular phone, portable tablet, personal computing device and cloud storage.

14. The method of claim 13, wherein the change in brain state is an epileptic event, and the brain state data comprises epileptic event data.

15. The method of claim 13, wherein the incision is less than 5 cm.

16. The method of claim 13, wherein the implantable body is inserted at an angle between 0 to 90° to the anterior posterior head axis of the patient.

17. The method of claim 13, wherein the implantable body includes a central axis bisecting the length of the implantable body, the first electrode contact being positioned proximal of the central axis and the second electrode contact being positioned distal of the central axis.

18. The method of claim 14, wherein the first and second electrode contacts are separated by a distance selected to form a single channel for detection of ictal activity with a signal to noise ration of at least 1.1 to 1.

19. The method of claim 14, further comprising:
administering an anti-seizure treatment to the patient when the processor detects ictal activity.

20. The method of claim 14, wherein the implantable body remains in vivo for a period of at least 3 days.
